(19) United States
(12) Patent Application Publication
(10) Pub. No.: US 2003/0073625 A1
(43) Pub. Date: Apr. 17, 2003

(54) Methods of preventing ventilator associated pneumonia by oral administration of antimicrobial IB-367 peptides

(76) Inventors: Rebecca Redman, San Jose, CA (US); Henry Fuchs, San Francisco, CA (US)

Correspondence Address: COOLEY GODWARD, LLP, 3000 EL CAMINO REAL, 5 PALO ALTO SQUARE, PALO ALTO, CA, 94306, US

(21) Appl. No.: 10/075,924

(22) Filed: Feb. 12, 2002

Related U.S. Application Data
(60) Provisional application No. 60/268,585, filed on Feb. 13, 2001.

(51) Int. Cl. A61K 38/17

(52) U.S. Cl. 514/12

(57) ABSTRACT

The present invention provides methods of preventing respiratory infections associated with intubation and/or mechanical ventilation, such as ventilator-associated pneumonia, in intubated patients. The method generally involves topical administration of a composition comprising an IB-367 peptide to the oral cavity of an intubated patient. As IB-367 peptides engender very little resistance, a significant advantage of the invention is that the prophylactic therapy may be applied without having to worry about creating resistant strains of pathogens.

### CROSS REFERENCE TO RELATED APPLICATIONS

This application claims benefit under 35 U.S.C. §119(e) to provisional application No. 60/268,585, filed Feb. 13, 2001. The contents of this provisional application are incorporated herein by reference.

### 1. FIELD OF THE INVENTION

The present invention relates to the prevention of respiratory infections commonly associated with intubation in patients requiring mechanical ventilation.

### 2. BACKGROUND OF THE INVENTION

Ventilator-associated pneumonia (“VAP”) is a common disorder among patients in intensive care units and long-term care facilities. It is associated with complications of intubation (insertion of an endotracheal tube) and mechanical ventilator support. Placement of an endotracheal tube allows bacteria to enter the lower respiratory tract directly and promotes microbial colonization by interference with the cough reflex, inhibition of mucociliary clearance and/or stimulation of excessive mucus secretion. The incidence of nosocomial, or hospital-acquired, pneumonia increases by as much as 6 to 20 times in patients receiving mechanical ventilatory support.

Approximately 880,000 patients are placed on ventilators in the United States each year, and between about 15-30% of patients who remain on mechanical ventilation for more than 48 hours develop VAP. Pneumonia is 6 to 21 times more frequent among patients receiving continuous mechanical ventilation than among those not receiving ventilator support. Nathens et al., 1999, “Selective Decontamination of the Digestive Tract in Surgical Patients: A Systematic Review of the Evidence,” Arch Surg 134:170-6; and Silvestri et al., 2000 “Selective Decontamination of the Digestive Tract: A Life Saver,”. J Hosp Infect 45:185-90. Pneumonia occurring within the first few days of ventilation, called early-onset VAP, pneumonia occurring after this period, referred to as late-onset VAP, are commonly caused by the aspiration of bacteria colonizing the patient's oropharynx and/or stomach or by primary entry into the lower respiratory tract. Crude mortality rates for VAP may be as high as 70 percent despite the availability of therapeutic agents and supportive care modalities. D'Amico et al., 1998, “Effectiveness of Antibiotic Prophylaxis in Critically Ill Adult Patients: Systematic Review of Randomised Controlled Trials,” BMJ 316:1275-85. It is estimated that the direct cost of treating nosocomial pneumonia, including VAP, in the U.S. is in excess of $1.1 billion per year. Wenzel, 1989, Eur J Clin Microbiol Infect 8:56-60.

Because bacteria colonizing the oropharynx and stomach are the primary cause of VAP, interventions to prevent VAP and its sequelae have targeted minimizing the bacterial colonization of the oropharynx and/or stomach. Selective decontamination of the digestive tract (SDD) is one prophylactic strategy designed to reduce bacterial colonization and decrease rates of respiratory tract infections. A variety of SDD regimens have been evaluated. Decontamination strategies include the application of antiseptics or antimicrobial agents directly to the surfaces of the oral cavity (selective oral decontamination, also referred to as selective oropharyngeal decontamination, or SOD) and/or solutions of these regimens delivered to the stomach by swallowing or through a feeding tube (selective gastric decontamination or SGD). In addition, many SDD regimens supplement the use of topical oral and/or gastric antimicrobial agents with the administration of intravenous antibiotics, particularly cefotaxime.

An analysis of the literature indicates that prophylaxis with SDD improves outcomes related to the development of pneumonia. Although SDD prophylaxis does not significantly reduce duration of mechanical ventilation, length of stay in the ICU, or length of stay in the hospital, it does significantly reduce the odds of death, particularly for regimens that include a systemic component. Prophylaxis with SDD reduces infections, including pneumonia, due both to gram positive and gram negative bacteria. Pneumonia occurs less frequently in SDD patients than control patients for many pathogens, including S. aureus and P. aeruginosa.

The propriety of SDD for the prevention of VAP is vigorously debated by the medical profession. The United States Centers for Disease Control and Prevention (CDC), in their Guideline for Prevention of Nosocomial Pneumonia, bluntly advises that currently available data do not justify the routine use of SDD for prevention of nosocomial pneumonia in ICU patients. In addition, the CDC cites concerns over the development of antimicrobial resistance and superinfection with gram-positive bacteria and other antibiotic-resistant nosocomial pathogens. Tablan et al., 1994, Infect. Control Hosp. Epidemiol. 15:587-627. In 1991, the European Consensus Conference in Intensive Care Medicine issued a recommendation that discouraged the systemic use of SDD in ventilated patients. Misset et al., 1996, Inten. Care. Med. 22:981-984. Indeed, at least one article concludes: “Selective decontamination of the digestive tract does not improve survival among patient receiving mechanical ventilation in the intensive care unit, although it substantially increases the cost of their care.” Gastinne et al., 1992, NEJM326:594-599.

New methods are needed to prevent respiratory infections and/or VAP in patients receiving mechanical ventilation, thus decreasing the time spent on the ventilator and increasing the patient's quality of life.

### 3. SUMMARY OF THE INVENTION

The present invention provides methods of preventing respiratory infections associated with intubation in patients receiving mechanical ventilation, such as ventilator-associated pneumonia. The methods may be practical in patients that are intubated orally, nasally or tracheally. According to the method, a composition comprising an antibiotic IB-367 peptide as an active ingredient is topically administered to the oral cavity of an intubated patient. Typically, the composition is applied directly to accessible surfaces of the oral cavity and to the visible portions of the endotracheal tube and retained for at least 1 -10 min., and preferably for at least 5 min., prior to rinsing. The composition may be applied prior to, concomitant with, or after the patient has been intubated. The composition may be optionally applied to the portions of the endotracheal tube that will be inserted into the patient to insure its sterility. The prophylactic therapy may be continued after the patient has been intubated as a means of delaying the onset of, or preventing altogether, ventilator-associated infections such as VAP. The resulting prevention of VAP will decrease overall cost of patient treatment and improve the quality of life in patients supported with mechanical ventilation.

### 4. BRIEF DESCRIPTION OF THE FIGURES

FIG. 1 provides a graph illustrating the effect of a single dose of native IB-367 oral rinse (containing 9 mg native 1B-367) on the total aerobic flora from the mouth, oropharynx and trachea of orally intubated patients;

FIG. 2 provides a graph illustrating the effect of a single dose of native IB-367 oral rinse (containing 9 mg native 18-367) oral Gram-positive and Gram-negative bacterial and yeasts of orally intubated patients;

FIG. 3 provides a graph comparing the effect of a single doses of native IB-367 oral rinse containing either 9 mg or 30 mg of native IB-367 on the total aerobic oral flora of orally intubated patients;

FIG. 4 provides a graph illustrating the effect of repeated dosing of native IB-367 oral rinse at Q 4 hr or Q 6 hr on the total aerobic oral flora of orally intubated patients; and

FIG. 5 provides a graph illustrating the mean-time plot of total aerobes from oral swabs of orally intubated patients treated Q 4 hr or Q 6 hr with 9 mg native IB-367.

### 5. DETAILED DESCRIPTION OF THE PREFERRED EMBODIMENTS

### 5.1 Antimicrobial IB-367 Peptides

The methods of the present invention utilize the administration of an antimicrobial IB-367 peptide. Native antimicrobial peptide IB-367 is a 17 amino acid residue synthetic protegrin peptide having the following amino acid sequence (listed in the N→C direction in accordance with common practice):

RGGLCYCRGRFCVCVGR (SEQ ID NO:1)

The peptide has four cysteine residues and can exist in any of a variety of disulfide-bridged forms. In preferred forms, the peptide has two disulfide bridges: one between Cys⁵ and Cys¹⁴ and another between Cys⁷ and Cys². Thus, while not intending to be bound by any particular theory, it is believed that native IB-367 adopts a disulfide-bridged β-sheet structure in solution. The native IB-367 peptide is amidated at the carboxyl terminus, although forms of the peptide having a free carboxyl terminus (“carboxy IB-367”) are also active. Moreover, while the native IB-367 is composed entirely of L-amino acids, the analog composed entirely of D-amino acids (“enantio IB-367”) is also active. As used herein the expression “IB-367” is intended to include the native, carboxy and enantio forms of the peptide. When a specific form is intended, it is preceded with the prefix “native,” “carboxy” or “enantio.”

IB-367 is ideally suited to preventing infections associated with oral intubation, such as ventilator-associated pneumonia (“VAP”), in animal patients, and particularly in human patients. IB-367 has broad spectrum activity against a variety of pathogens, including the Gram-positive and Gram-negative bacteria that are frequently associated with VAP. For example, IB-367 is microbicidal against, amongst others, S. aureus, including methicillin-resistant S. aureus (“MRSA”), P. aeruginosa, Acinetobacter sp. and Klebsiella sp, as well as other pathogens associated with VAP. Moreover, native peptide IB-367 retains its microbicidal activity in human saliva, rapidly killing the polymicrobial flora of the oral cavity. The beneficial features of IB-367, including rapid microbicidal action in saliva against relevant pathogens, a broad spectrum of action and low propensity to engender resistance, make IB-367 peptides ideally suited for preventing and/or reducing the incidence of infections associated with intubation and/or mechanical ventilation, such as VAP.

The protegrin peptides generally have been shown to engender very little resistance. For example, repeated subcultures of bacteria in the presence of protegrin peptide PG-1 at one-half minimum inhibitory concentration (MIC) did not result in the development of resistance. See Steinberg et al., 1997, Antimicrob. Agents and Chemother. 41(8):1738-1742. Thus, unlike conventional antibiotics, which engender resistance and are therefore not appropriate for prophylactic use, IB-367 peptides are ideally suited for the prophylactic treatment of intubated patients in an effort to delay the onset of, or prevent altogether, infections associated with such intubation. Owing to the fact that they do not engender resistance, these peptides may be used prophylactically without fear of creating strains of pathogens resistant to treatment. In addition, prophylactic use of IB-367 may avoid the introduction of resistant organisms into the ICU and other hospital environs.

Native IB-367, as well as its related carboxy and enantio forms, may be conveniently synthesized using standard solid phase or solution phase peptide synthesis methodologies. Specific methods for synthesizing the IB-367 peptides, as well as for oxidizing the IB-367 peptides to form the various disulfide bridged forms, are described, for example, in U.S. Pat. No. 5,804,558 and U.S. Pat. No. 5,994,306, the disclosures of which are incorporated herein by reference.

### 5.2 Administration

The methods of the invention generally involve topically applying to the oral cavity of an intubated subject an amount of an IB-367 peptide effective to prevent respiratory infections associated with intubation and/or mechanical ventilation, such as VAP. While not intending to be bound by any theory of operation, it is believed that insertion of the endotracheal tube permits bacteria to enter the lower respiratory tract. Moreover, it is thought that the tube directly promotes microbial colonization by interfering with the cough reflex, by inhibiting mucociliary clearance and/or by stimulating excessive mucus secretion. Thus, the ability to rapidly and broadly lower the bacterial load in the oral and oropharyngeal cavities through application of antimicrobial IB-367 peptides provides a means of preventing respiratory infections associated with, or caused by, intubating patients.

As used herein, SOD, or selective oral decontamination, refers to topical application of antimicrobial agents to accessible surfaces of the oral cavity. The term SGD, or selective gastric decontamination means administering solutions of antimicrobial agents to the stomach by swallowing or through a feeding tube. Selective digestive decontamination (SDD), properly, refers to the combination of both SOD and SGD, thus decontaminating the entire pre-digestive tract, including oropharyngeal and gastric regions. Any of the selective decontamination regimens, SOD, SGD or SDD, may further be supplemented by the administration of systemic antibiotics aimed at eliminating microbial pathogens of the digestive tract. In one embodiment, the prophylactic regimen involves SOD.

An effective dose refers to that amount of peptide sufficient to delay the onset of, or avoid altogether, the development of a respiratory infection and/or VAP in intubated patients. In one embodiment, an effective dose is that amount of peptide sufficient to reduce in the oral cavity of the subject the number colony forming units (“CFUs”) of flora associated with the infection as compared to the number of CFUs observed prior to treatment. Typically, a reduction of CFUs on the order of 3-4 log is considered to be effective; however, even reductions on the order of 1-2 log may provide significant prophylaxis.

Those of skill in the art will recognize that prophylaxis as used herein does not exclude the possibility that the respiratory system or lungs of the patient may become colonized with bacteria, whether from the oral cavity or from other sources. Rather, prophylaxis as used herein means that such colonization either does not lead to a diagnosed respiratory infection or pneumonia using standard, well-known diagnostic criteria, or that the onset of such an infection or pneumonia is delayed as compared to intubated patients not receiving the therapy. In this latter context, the prophylactic therapy provides significant benefit in patients who are intubated or who receive mechanical ventilation for relative short durations of time. The prophylactic therapy may be applied to intubated patients regardless of the type of intubation used. For example, the therapy may be applied to patients that are orally intubated, nasally intubated or in patients receiving tracheal ventilation.

For any IB-367 peptide, an effective dose can be estimated initially from in vitro tests such as, for example, MICs and saliva kill kinetics. Initial dosages can also be estimated from in vivo data, e.g., animal models, using techniques that are well known in the art. One having ordinary skill in the art could readily optimize administration to humans based on animal data, especially in light of the detailed disclosure herein. The CFUs in the oral cavity of the patient can be conveniently obtained (such as by swab tests) to monitor the efficacy of the therapy.

In general, the peptides will be most beneficial when applied to the oral cavity of an intubated patient before the onset of a respiratory infection such as VAP. Thus, in general, treatment will begin concomitant with, just prior to, or shortly after intubation of the patient, and may be continued until the patient is removed from the ventilator. Of course, whether and when to begin and end treatment will depend upon the judgement of the treating physician.

As will be discussed in more detail below, the peptide(s) will typically be administered in the form of a topical oral composition or formulation. Such formulations will generally comprise about 0.001% (w/w) to about 2.5% (w/w) total peptide; however, concentration ranges such as about 0.005% (w/w) to about 0.75% (w/w) or even about 0.03% (w/w) to about 0.3% (w/w) are expected to be effective. In one embodiment, the composition is non-absorble such that the IB-367 peptides are not appreciably absorbed by the tissues contacted. Such non-absorbable compositions avoid systemic uptake and permit local administration directly to the site of contamination.

The composition may be applied topically several times per day, depending in part upon the concentration of the applied dose and other factors such as the frequency of food and fluid intake by the patient. Thus, depending on the particular circumstances, the composition may be applied 1, 2, 3, 4 or even as many as 6 times per day. The treatment may be administered for a single day, for several days (e.g., from 2-5 or more days), for several weeks or for the entire period during which the patient is intubated. The duration of the treatment will depend upon the duration of intubation and thus on the period of risk for developing VAP and may be decided by the treating physician. For example, it may be applied for the entire period during which the patient is intubated, or for such other period that the treating physician judges the patient is at an elevated or high risk of developing a respiratory infection associated with intubation, such as VAP.

The treatment may include adjunctive administration of systemic antibiotics. By “adjunctive administration” is meant that the systemic treatment may be applied prior to, concomitant with, or after treatment with the topical application of the IB-367 peptide. In this context, a systemic antibiotic is selected that is thought to be useful for SOD and/or SDD therapeutic approaches to treat or prevent VAP. Examples of such antibiotics include, but are not limited to, cefotaxime, ceftazidime, cefazolin, cephradine, cefuroxime, ciprofloxacin, vancomycin, tobramycin, ampicillin, piperacillin, carbenicillin, tricarcillin, metronidazole, erythromycin, gentamycin, trimethoprim, clindamycin, tetracycline, tazobactam, linezolid and trimethoprim-sulfamethoxazole.

In one embodiment, the IB-367 treatment excludes the adjunctive use of systemic antibiotics aimed at treating or preventing VAP. In this embodiment, the IB-367 peptide is applied topically as described above without the aid of antibiotics useful for SOD or SDD. Of course, those of skill in the art will recognize that systemic antibiotic treatments aimed at combating other, non-VAP infections, may still be applied.

The actual mode of administration will depend upon the choice composition or formulation, e.g., whether the composition is a rinse or gel, and will be common for the type of composition or formulation being applied. Preferably, care should be taken to avoid aspiration of the composition. Liquid compositions such as rinses, syrups, elixirs, emulsions, etc. may be conveniently applied to the accessible surfaces of the oral cavity, and optionally on the accessible surfaces of the intubation tube, using an applicator such as a sponge or other soft, absorbant applicator. Alternatively, these formulations may be sprayed onto the accessible surfaces of the oral cavity, and optionally on the accessible surfaces of the intubation tube with the aid of a sprayer. The sprayer may be a mechanical pump sprayer or other type of sprayer commonly employed in the art. The treatment should be allowed to reside in the mouth for a time sufficient to kill the resident pathogens, for example on the order of about 1-10 min., preferably for about 5 min., before rinsing the oral cavity.

### 5.3 Compositions

As discussed above, the IB-367 peptide is administered topically to the oral cavity of an intubated patient, or a patient about to be intubated, in the form of an oral composition. Virtually any composition that may be administered or applied topically to the oral cavity of an animal patient, especially a human patient, may be used. Such compositions and formulations are well-known in the art and include, by way of example and not limitation, oral solutions, oral syrups, oral elixirs, oral suspensions, oral emulsions, oral sprays, oral lozenges, oral magmas and oral gels. Examples of such compositions may be found, for example, in Ansel et al., 1995, Pharmaceutical Dosage Forms and Drug Delivery Systems, A Lea & Febiger Book, Williams & Wilkins, Malvern, Pa., pp. 227-285. A specific example of an oral gel suitable for use in the methods of the invention is found in U.S. Pat. No. 6,025,326, incorporated herein by reference, although the gel formulation is less effective than the rinse formulation described below and in the illustrative examples.

The IB-367 peptide may be included in such topical oral compositions in the form of a free acid, a free base or as a pharmaceutically acceptable salt, as are well-known in the art. Typically, pharmaceutically acceptable salts are preferred for aqueous oral compositions such as rinses and swishes, as these peptide salts tend to be more soluble than the free acid or base forms. A particularly preferred pharmaceutically acceptable salt is the hydrochloride salt.

As discussed above, the peptide is typically included in the composition in an amount effective to achieve the desired prophylactic effect, in a single application per day, although more typically multiple applications per day may also be used.

The composition may comprise a single IB-367 peptide, such as native IB-367, or a combination of IB-367 peptides, such as native IB-367 and enantio IB-367. Moreover, the composition may comprise additional active ingredients, such as conventional antibiotics, antifungals, antivirals or pain killers, etc.

Preferred compositions for topical oral administration are aqueous-based oral rinses or swishes comprising about 0.3 wt % total IB-367 peptide. Such compositions may include additional ingredients, such as humectants, sweeteners, mucoadhesives, buffers, preservatives, etc., as are well-known in the art. Typically, such compositions will have a pH in the range of about pH 3-5, with a preferred pH being about pH 4.0, although any pH that does not deleteriously affect the activity of the peptide and that is suitable for topical oral administration may be used. A particularly preferred oral rinse composition is provided in Table 1, below:

TABLE 1

| Ingredient | Amount | Purpose |
|---|---|---|
| Native IB-367¹ | 0.3 wt %² | active |
| | | ingredient |
| Sorbitol | 10.0 wt % | humectant |
| Xylitol | 3.0 wt % | sweetener |
| HPMC³ | 0.2 wt % | mucoadhesive |
| Lactic acid | 0.1 wt % | buffer |
| Methyl Paraben | 0.18 wt % | preservative |
| Propyl Paraben | 0.02 wt % | preservative |
| Sodium hydroxide | q.s to pH 4.0 (±0.2) | pH adjustment |
| Hydrochloric acid | q.s. to pH 4.0 (±0.2) | pH adjustment |
| Purified water | balance to 100 wt % | solvent |

¹Supplied as the hydrochloride salt.
²Based on the weight of the anhydrous, free base form of the peptide.
³Hydroxypropyl methylcellulose.

The topical oral compositions or formulations may be prepared by any of a variety of art-known techniques. Preferably, the IB-367 peptide is added in the form of the hydrochloride salt, as the free base form of the peptide is sparingly soluble in water and tends to form a gel above concentrations of 20 mg/mL. The peptide should be formulated at concentrations below 15-20 mg/mL to avoid gelling and at low tonicity or in an isotonic solution. Notably, the viscosity of the composition may be readily adjusted by adding more or less HMPC, as the amount of HMPC does not detrimentally affect the stability of the peptide. Moreover, the IB-367 peptide should be added to the composition at temperatures which will not degrade or otherwise deleteriously affect the activity of the peptide, such as temperatures in the range of about 40-44° C., or lower.

### 6. EXAMPLES

The following examples are intended to illustrate, and not limit, the invention.

### 6.1 Preparation of Native IB-367 Oral Rinse Formulation

A typical 75 kg batch of the native IB-367 oral rinse formulation described in Table 1 is prepared as follows. Lactic acid (75 g) is dissolved into an appropriate amount of purified water and the pH of the solution is adjusted to pH 4.0 (±0.2) with 1 N NaOH and/or 1 N HCl solution. The resultant solution is heated to 72-78° C. The methyl paraben (135 g) and the propyl paraben (15 g) are added and mixed until completely dissolved. The HMPC (150 g) is slowly added, stirring rapidly. The solution is stirred while cooling until the temperature of the solution reaches 50-60° C. The mixing speed is reduced to avoid aeration, and the sorbitol (7500 g) solution is added and mixed. The xylitol (2250 g) is added and dissolved. The solution is slowly mixed (to avoid aeration) while continuing to cool until the temperature reaches between 40-44° C. Native IB-367 (225 g) is then added to the mixture with continuous mixing. Purified water is added to bring the mixture to the final weight, and the solution is slowly mixed (to avoid aeration) until homogeneous. The pH of the solution is checked and, if needed, the pH is adjusted to pH 4.0 (±0.2) with 1 N NaOH and/or 1 N HCl solution. The composition is then dispensed into single-dose containers.

Placebo Rinse is prepared as described above without the addition of native IB-367. One mL of the composition weighs approximately one gram. Different batch sizes may be prepared using proportional quantities of ingredients. In addition, batches including higher or lower amounts of active ingredient may be made by adjusting the amount of the active ingredient and water added. Moreover, batches comprising carboxy or enantio 18-367, or mixtures of native, carboxy and/or enantio IB-367 may be prepared by the same method.

The 25° C. shelf-life of the composition is estimated to be greater than 2 yrs. at a pH in the range of pH3to 5.

### 6.2 A Single Dose of Oral Rinse Safely and Rapidly Reduced the Total Microbial Burden in Orally Intubated Patients

This Example illustrates that a single dose of native IB-367 oral rinse is active against Gram-positive and Gram-negative bacteria and yeasts in orally intubated patients, and showed no serious adverse events. The reductions in oral flora observed in both the oral and oropharyngeal cavities of intubated patients demonstrates the utility of IB-367 rinse in preventing ventilator-associated respiratory infections such as VAP.

### 6.2.1 Experimental Protocol

In a multicenter trial, single doses of native IB-367 rinse or a placebo control (as described in Table 1 and including either 0.3 wt % or 1 wt % native IB-367) were administered to sixteen orally intubated patients. Eight patients were randomized to receive a single dose of 9 mg native IB-367 Rinse (n=6) or a single dose of placebo Rinse (n=2). Eight additional patients were randomized to receive a single dose of 30 mg native IB-367 Rinse (n=6) or placebo Rinse (n=2). For all sixteen patients, 3 mL of IB-367 Rinse or placebo Rinse was applied directly to all surfaces of the oral cavity and to all visible surfaces of the endotracheal tube with a sponge applicator. The Rinse was retained for at least five minutes.

To measure the total oral microbial burden of each patient, oral swabs, oropharyngeal swabs and tracheal secretions were collected predose and at 0.5, 2, 4, 6, 8, 12, 18 and 24 hours following dosing.

### 6.2.2 Results

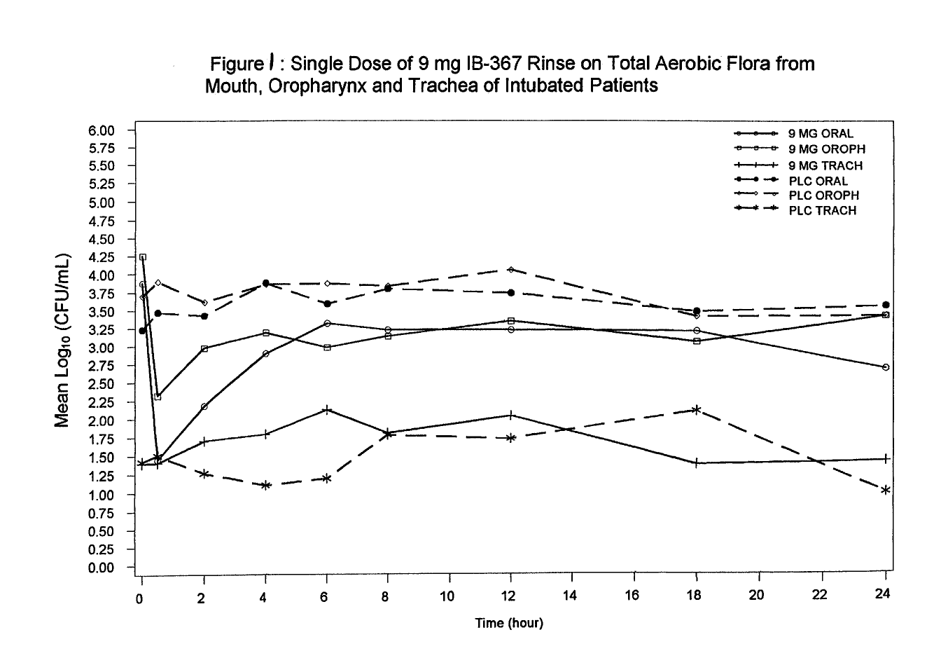

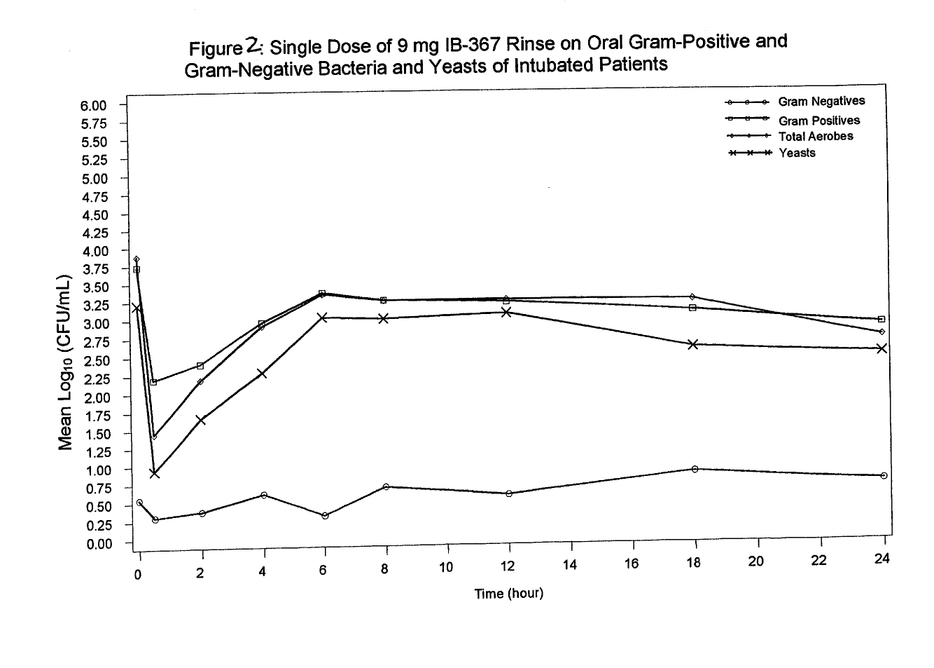

The total microbial burden of intubated patients that received a dose of native IB-367 Rinse was significantly reduced. For instance, as shown in FIG. 1 at 0.5 hours post dose, 9 mg native IB-367 Rinse significantly reduced the total oral aerobic flora of intubated patients (P=0.007). In addition, as shown in FIG. 2, a single dose of 9 mg native IB-367 Rinse reduced the total oral gram-positive bacteria, the total gram-negative bacteria and the total oral yeast of intubated patients.

Furthermore, as shown in FIG. 1, a single dose of 9 mg native IB-367 Rinse produced a reduction of the oropharyngeal microbial burden of intubated patients similar to that of the reduction of the oral microbial burden, albeit at a smaller magnitude.

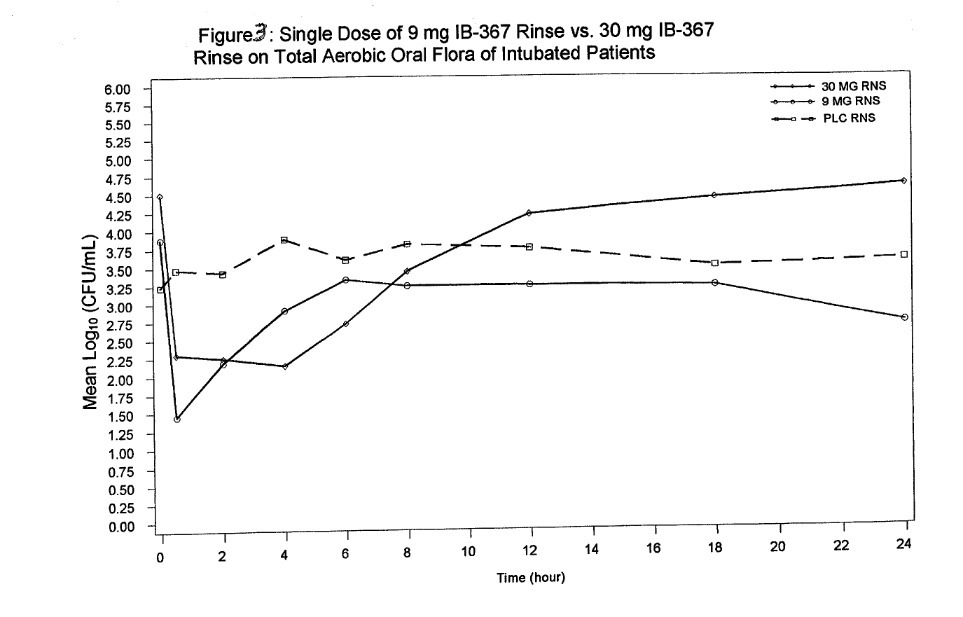

As shown in FIG. 3, a single dose of 30 mg native IB-367 Rinse produced a decrease in the total aerobic oral flora of intubated patients that was similar in magnitude and duration to the decrease produced by a single dose of 9 mg native IB-367 Rinse.

No serious adverse event related to the administration of native IB-367 Rinse was observed.

### 6.3 Repeated Dosing of IB-367 Reduced the Total Microbial Burden in Orally Intubated Patients

This Example illustrates that regular periodic administration of native IB-367 oral rinse over a five day period is active against Gram-positive and Gram-negative bacteria and yeasts in orally intubated patients, and showed no serious adverse events.

### 6.3.1 Experimental Protocol

In a multicenter trial, single doses of native IB-367 rinse (as described in Table 1 and including either 0.3 wt % native IB-367) were administered to sixteen orally intubated patients. Eight patients were randomized to receive 9 mg native IB-367 Rinse (n=6) or placebo Rinse (n=2), applied to the oral cavity every six hours for five days. Eight additional patients were randomized to receive a 9 mg native IB-367 Rinse (n=6) or placebo Rinse (n=2), applied to the oral cavity every four hours for five days. For all sixteen patients, 3 mL of IB-367 Rinse or placebo Rinse was applied directly to all surfaces of the oral cavity and to all visible surfaces of the endotracheal tube with a sponge applicator. The Rinse was retained for at least five minutes, then was suctioned, swallowed or expectorated.

Oral swabs and tracheal secretions were collected before the first dose of each treatment day, 30 min after the first dose of each treatment day, and once daily for the duration of intubation. Oral and tracheal samples were processed at a central laboratory for quantitative microbial analysis, identification, and susceptibility testing. Blood was collected and analyzed for plasma IB-367 concentration at three time points (before the first dose, 15 min after the first dose, and 15 min after the last dose). Blood was also collected and analyzed for antibodies to IB-367 at two time points (pre-dose and at study exit).

### 6.3.2 Results

Sixteen patients were randomized, they all received treatment and their data were analyzed. Seven patients had study medication discontinued before the completion of 5 days of therapy. Early extubation was the reason for six of the seven discontinuations. One patient discontinued due to an adverse event, a rash.

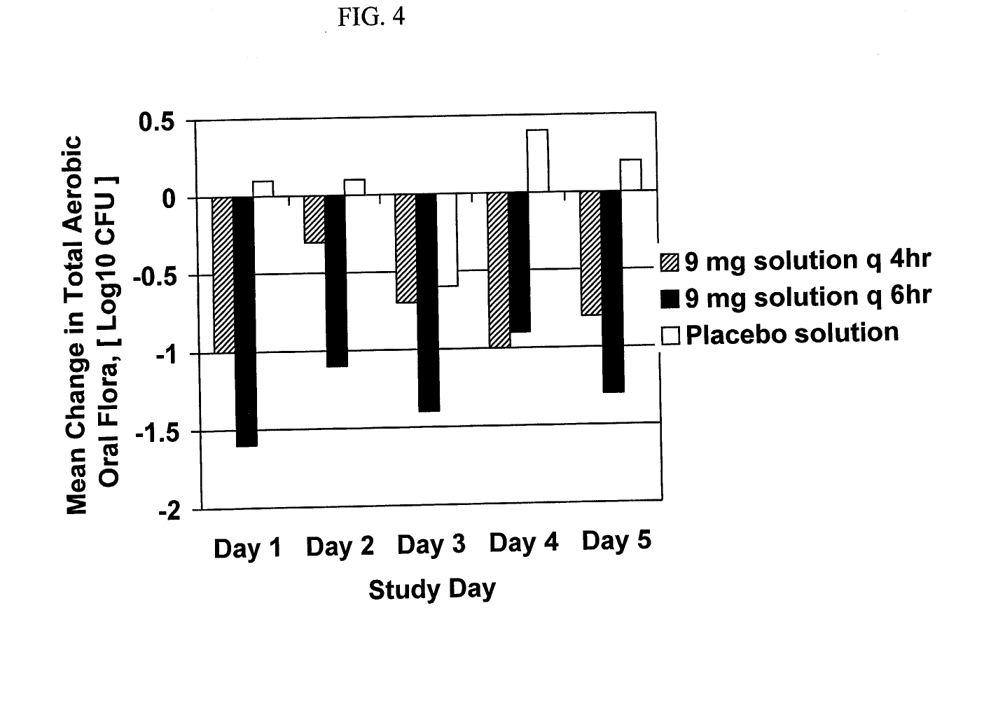

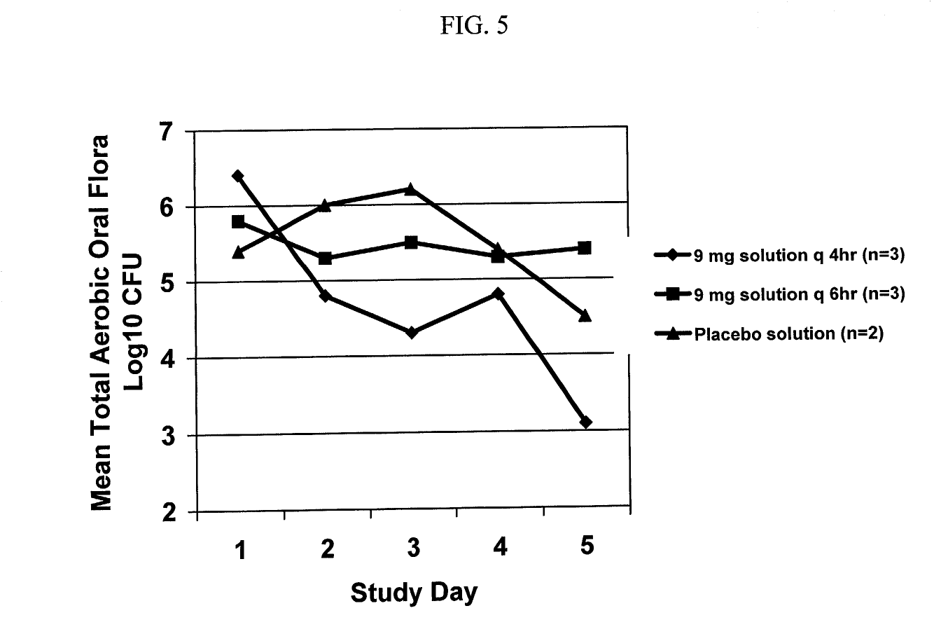

The total microbial burden of intubated patients that received the native IB-367 Rinse was significantly reduced as early as the first day. For instance, as shown in FIG. 4 at Day One, patients receiving the IB-367 Rinse experienced a 10-fold decrease (Q 4hr dosing) or a an approximately 30-fold decrease (Q 6hr dosing) in total aerobic oral flora. Further, as shown in FIG. 5, the patients receiving native IB-367 Rinse also experienced a cumulative decrease in oral bio-burden over the five date treatment regimen.

Of the concentrations and formulations tested in this study, 9 mg IB-367 rinse formulation, given Q4H, provided the greatest anti-microbial effect in terms of immediate reduction and cumulative effect in orally intubated and mechanically ventilated patients. Based on the efficacy and safety results observed in this study, IB-367 rinse formulation is a promising broad spectrum single agent candidate for the prevention of ventilator-associated respiratory infections, including VAP.

While the invention has been described by reference to various specific embodiments, skilled artisans will recognize that numerous modifications may be made thereto without departing from the spirit and scope of the appended claims.

All references cited throughout the disclosure are incorporated herein by reference in their entireties for all purposes.

What is claimed is:

1. A method of preventing a respiratory infection associated with intubation or mechanical ventilation, comprising topically applying to the oral cavity of a patient that is either intubated or about to be intubated a composition comprising an IB-367 peptide or a pharmaceutically acceptable salt thereof, said composition being applied in an amount effective to prevent the infection.

2. The method of claim 1 which consists essentially of selective oral decontamination.

3. The method of claim 1 in which the composition is an oral rinse.

4. The method of claim 1 in which the composition comprises about 0.03 wt % to about 0.3 wt % IB-367.

5. The method of claim 4 in which the composition is applied once or multiple times per day for 2 to 5 days.

6. The method of claim 4 in which 3 mL of the composition is applied once every 4 to 6 hours.

7. The method of claim 1, 2, 3, 4, 5, or 6 in which the IB-367 is native IB-367.

8. The method of claim 1 in which the composition is also applied to the accessible portions of an endotracheal tube that either is or will be inserted into the patient.

9. The method of claim 1 in which the respiratory infection is VAP.

10. A method of preventing VAP, comprising topically applying to the oral cavity of a patient that is at risk of developing VAP an aqueous composition comprising: 0.3 wt % native IB-367 peptide or a pharmaceutically acceptable salt thereof, 10 wt % sorbitol, 3 wt % xylitol, 0.2 wt % hydroxypropyl methylcellulose, 0.1 wt % lactic acid, 0.18 wt % methyl paraben, 0.02 wt % propyl paraben and water to balance and which has a pH of about 4, said composition being applied in an amount effective to prevent VAP.

11. The method of claim 10 which consists essentially of selective oral decontamination.

12. The method of claim 10 in which a single 3 ml dose is applied per day.

13. The method of claim 10 in which the composition is applied for 2 to 5 days.

14. The method of claim 10 in which 3 ml of the composition is applied once every 4 to 6 hours.

15. The method of claim 10, 12, 13 or 14 in which the native IB-367 is in the form of a hydrochoride salt.

16. The method of claim 10 in which the composition is also applied to the visible portions of an endotracheal tube that either is or will be inserted into the patient.

17. A method of preventing respiratory infection associated with intubation or mechanical ventilation, consisting essentially of topically applying to the oral cavity of a patient that is either intubated or about to be intubated a composition comprising an IB-367 peptide or a pharmaceutically acceptable salt thereof, said composition being applied in an amount effective to prevent the respiratory infection.

18. The method of claim 17 in which the composition is an oral rinse.

19. The method of claim 17 in which the composition comprises about 0.03 wt % to about 0.3 wt % IB-367.

20. The method of claim 19 in which the composition is applied once or multiple times per day for 2 to 5 days.

21. The method of claim 19 in which 3 mL of the composition is applied once every 4 to 6 hours.

22. The method of claim 17, 18, 19, 20 or 21 in which the IB-367 is native IB-367.

23. The method of claim 17 in which the composition is also applied to the accessible portions of an endotracheal tube that either is or will be inserted into the patient.

24. The method of claim 17 in which the respiratory infection is VAP.
